(19) United States
(12) Patent Application Publication
(10) Pub. No.: US 2025/0276969 A1
(43) Pub. Date: Sep. 4, 2025

(54) 2-(5-FLUORO-1-(2-FLUOROBENZYL)-1H-PYRAZOLO[3,4-b]PYRIDIN-3-YL)-5-NITROSOPYRIMIDIN-4,6-DIAMINE OR A SALT THEREOF, METHOD FOR THE PREPARATION THEREOF, AND USE THEREOF IN THE SYNTHESIS OF VERICIGUAT

(71) Applicant: MOEHS IBERICA, S.L., Rubí, Barcelona (ES)

(72) Inventors: Carles SÁNCHEZ CASALS, Rubí, Barcelona (ES); Alicia DOBARRO RODRÍGUEZ, Rubí, Barcelona (ES); Jordi GONZÁLEZ GARCÍA, Rubí, Barcelona (ES)

(21) Appl. No.: 18/861,251

(22) PCT Filed: Apr. 17, 2023
(86) PCT No.: PCT/EP2023/059928

§ 371 Date: Oct. 29, 2024

(30) Foreign Application Priority Data
May 3, 2022 (ES) P202230403

(51) Int. Cl. C07D 471/04

(52) U.S. Cl. CPC C07D 471/04

(57) ABSTRACT

The invention is directed to a 2-(5-fluoro-1-(2-fluorobenzyl)-1H-pyrazolo[3,4-b]pyridin-3-yl)-5-nitrosopyrimidin-4,6-diamine, or a salt thereof, to a method for its preparation, to the use of said compound in the preparation of vericiguat, to a new salt of vericiguat and trifluoroacetic acid, to the preparation of said salt of vericiguat and trifluoroacetic acid and to a process of preparing vericiguat from the trifluoroacetic acid salt of vericiguat.

### FIELD OF THE INVENTION

The present invention relates to a novel intermediate useful in the synthesis of vericiguat, to a method for obtaining same, to the use of said intermediate for the preparation of vericiguat, and to a method for the preparation of vericiguat using said intermediate.

### BACKGROUND OF THE INVENTION

Vericiguat is a soluble guanylate cyclase (GC) stimulator active pharmaceutical ingredient indicated for the treatment of symptomatic chronic heart failure in adults with a reduced ejection fraction who are stabilized after a recent episode of decompensation which required intravenous treatment. Vericiguat is a compound of formula (IV)

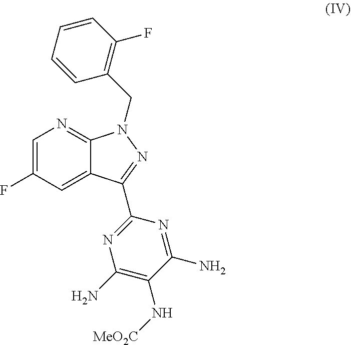 [SMILES: COC(=O)Nc1c(N)nc(-c2nn(Cc3ccccc3F)c3ncc(F)cc23)nc1N]

Said active ingredient acts by re-establishing NO-GC-cGMP signaling pathway relative deficiency by means of the direct stimulation of GCs, independently and synergistically with NO (nitric oxide), in order to increase intracellular cGMP (cyclic guanosine monophosphate) levels, which can improve both myocardial and vascular functions. Vericiguat was approved for medical use in the United States in January 2021 and in Europe in August 2021.

Several synthetic pathways have been described for the preparation of vericiguat (IV), see EP2576547B1 and EP2782914B1, for example.

EP2576547B1 describes several alternative methods for the preparation of vericiguat. A first method comprises the following synthetic steps:

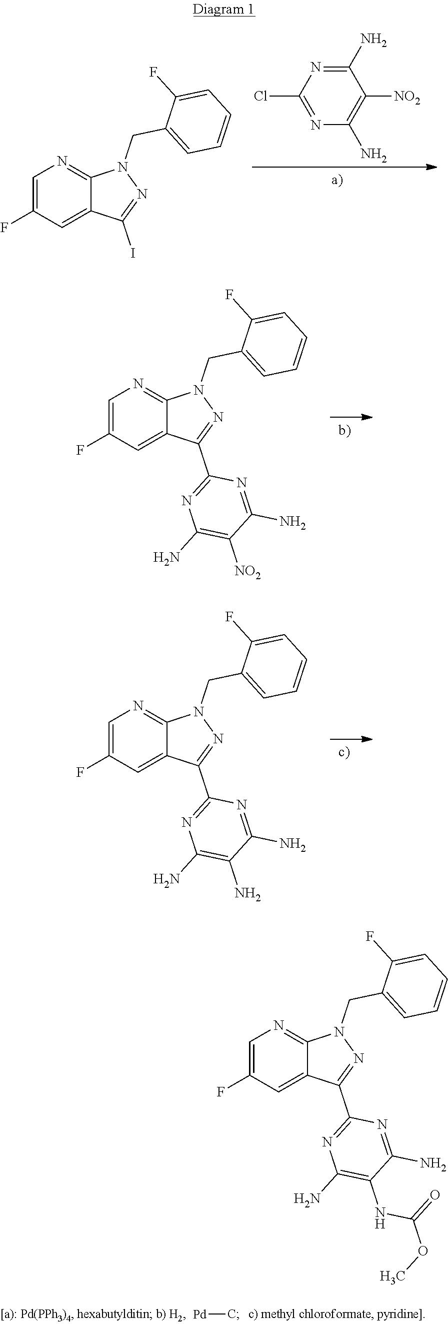

Nevertheless, the yield obtained in said synthesis is not mentioned. An alternative method for the synthesis of vericiguat which requires the preparation of an amidinium acetate salt as a precursor for the formation of a pyrimidine ring by means of a reaction with a malononitrile derivative is also described, such as in Diagram 2.

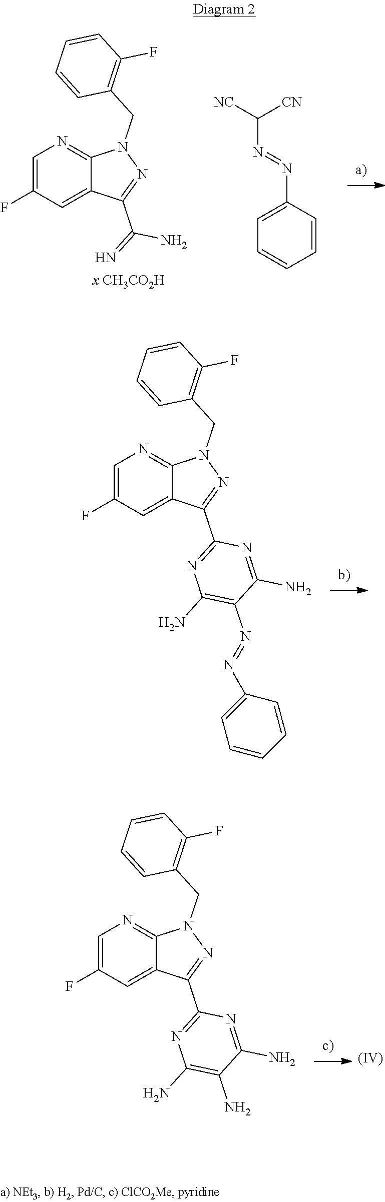 [SMILES: N=C(N)c1nn(Cc2ccccc2F)c2ncc(F)cc12 ; N#CC(C#N)/N=N/c1ccccc1 ; Nc1nc(-c2nn(Cc3ccccc3F)c3ncc(F)cc23)nc(N)c1/N=N/c1ccccc1 ; Nc1nc(-c2nn(Cc3ccccc3F)c3ncc(F)cc23)nc(N)c1N ; "(IV)"]

It is described that the product of synthetic step a) of diagram 2 is obtained with a 59% yield. The step of preparing free amine by means of the catalytic hydrogenation of the phenyldiazine derivative is reported with a quantitative yield and the step of forming vericiguat by means of reacting the amine with methyl chloroformate is reported with a 65% yield. Therefore, this document describes the synthesis of vericiguat from an aminidinium acetate salt with an overall yield of 38% from the 3 preparation steps. Vericiguat is isolated by means of precipitation in diethyl ether. This document also describes a method for the preparation of vericiguat salts, such as hydrochloride, sulfonate, phosphonate, maleate, among others, by means of reacting vericiguat with the corresponding acid (hydrochloric, sulfuric, phosphoric, and maleic acids).

Document EP2782914B1 discloses a method for obtaining vericiguat based on the reaction of a hydrochloride salt of an amidinium derivative with a previously prepared malononitrile derivative for the formation of the pyrimidine ring present in vericiguat. Said formation of the pyrimidine ring occurs with a yield of between 82% and 86% depending on the method used. The next step of reducing the phenyldiazine group to amino group by means of palladium on carbon-mediated catalytic hydrogenation is described with a yield of 80.3%. Vericiguat is formed by means of the reaction of the amine thus formed with methyl chloroformate or dimethyl dicarbonate. The crude product thus obtained is purified by means of a solvation process with dimethylsulfoxide (DMSO). The combined yield from solvate production and subsequent desolvation is 65%.

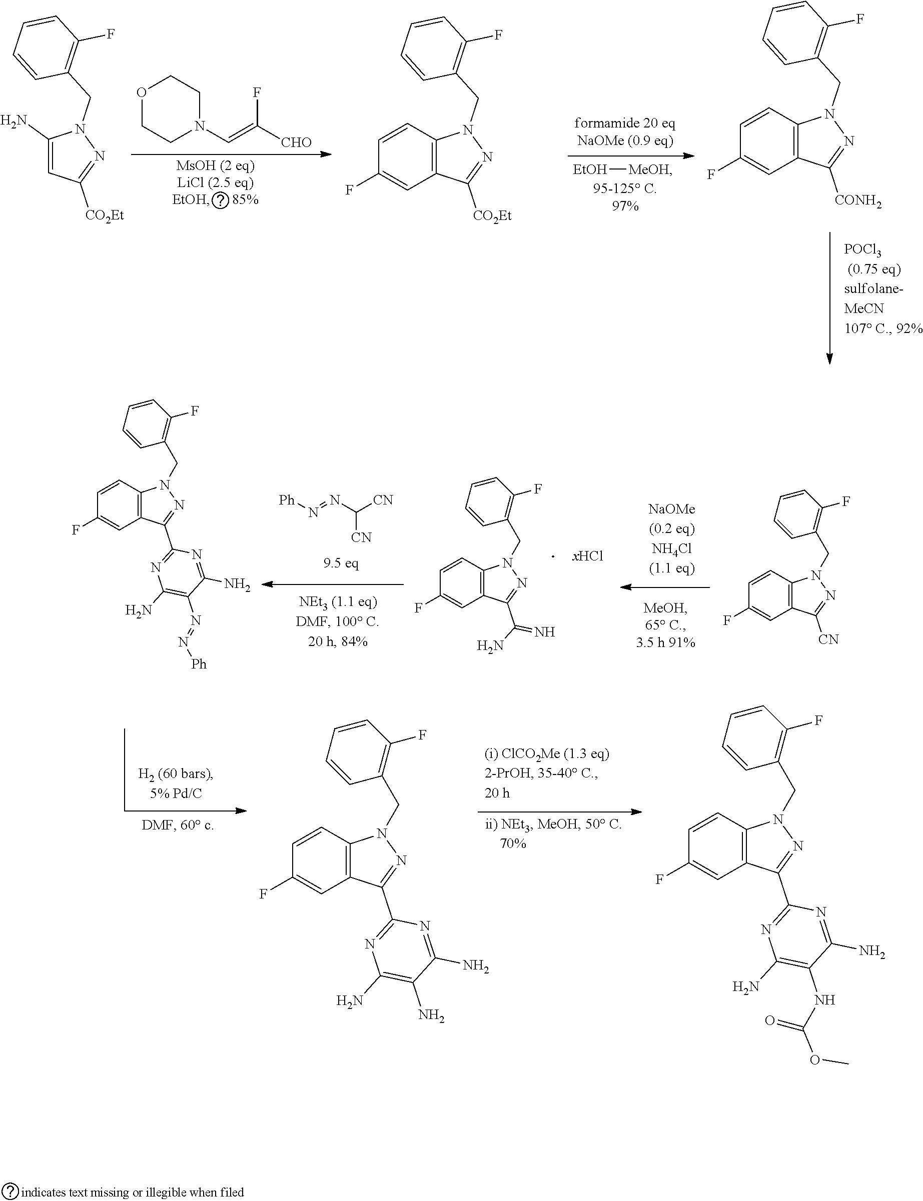

Document EP1102768B1 describes the reaction of a non-fluorinated amidine derivative with the sodium hydroxyiminomalononitrile sodium salt to form a pyrimidine ring with a nitrous functional group:

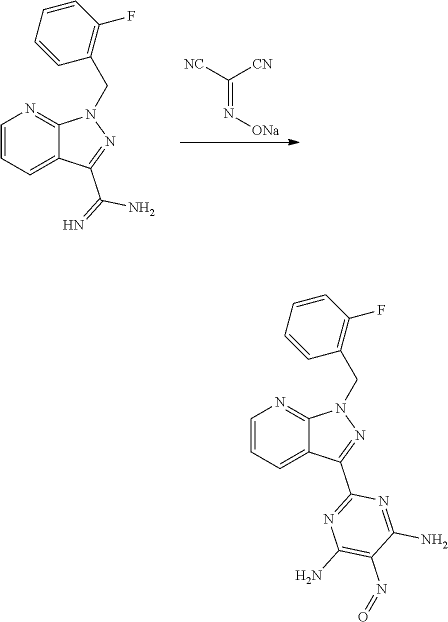

A yield of 1.8% is disclosed for this reaction. Therefore, this document teaches that this pathway is not suitable if an optimum yield is sought for the synthetic method.

A complete synthesis of vericiguat, similar to that described in EP2782914B1, was reported in Synfacts 2017, 13(09), 0897.

Document CN115160312A (which was filed on 29 Jun. 2022 and published on 11 Oct. 2022, i.e. after the priority date of the present application) describes a method for obtaining compound of formula (I).

Methods for obtaining vericiguat with a reduced number of synthetic steps and/or a higher overall synthetic yield are needed in the state of the art.

### SUMMARY OF THE INVENTION

The inventors have discovered a novel intermediate for the synthesis of vericiguat which allows vericiguat to be prepared in a reduced number of synthetic steps and with a higher yield.

In particular, the intermediate for the synthesis of vericiguat of the invention allows vericiguat to be prepared in two synthetic steps from an amidinium hydrochloride-type intermediate (compound of formula (II)) described in the art and with a yield greater than 65%. Compound of formula (II) may be prepared by the process described in example 9 of EP2782914B1.

To that end a first aspect of the invention relates to a compound of formula (I), which is 2-(5-fluoro-1-(2-fluorobenzyl)-1H-pyrazolo[3,4-b]pyridin-3-yl)-5-nitrosopyrimidin-4,6-diamine, or a salt thereof

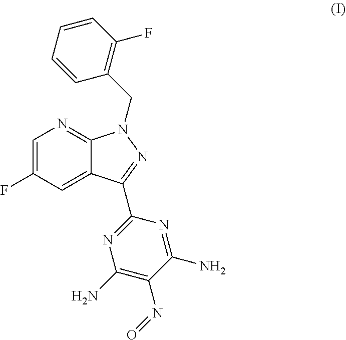 [SMILES: Nc1nc(-c2nn(Cc3ccccc3F)c3ncc(F)cc23)nc(N)c1N=O]

In a second aspect, the invention relates to a method for the preparation of a compound of formula (I) or a salt thereof as defined in the first aspect of the invention, characterized in that it comprises reacting a compound of formula (II), which is 5-fluoro-1-(2-fluorobenzyl)-1H-pyrazolo[3,4-b]pyridin-3-carboximidamide, or a salt thereof, with a salt of formula (III)

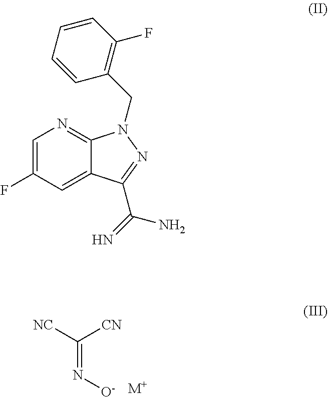

wherein M is an alkali metal.

A third aspect of the invention relates to the use of the compound of formula (I) or a salt thereof as defined in the first aspect of the invention in the preparation of vericiguat, which is methyl (4,6-diamino-2-(5-fluoro-1-(2-fluorobenzyl)-1H-pyrazolo[3,4-b]pyridin-3-yl)pyrimidin-5-yl) carbamate, or a salt thereof.

A fourth aspect of the invention relates to a method for the preparation of vericiguat, which is methyl (4,6-diamino-2-(5-fluoro-1-(2-fluorobenzyl)-1H-pyrazolo[3,4-b]pyridin-3-yl)pyrimidin-5-yl)carbamate, or a salt thereof, which comprises:
(a) reacting the compound of formula (I) or a salt thereof as defined in the first aspect of the invention in order to convert the nitrous group into an amino group; and
(b) reacting the compound resulting from step (a) in order to convert the amino group formed from the nitrous group of the compound of formula (I) or the salt thereof into a group of formula —NHCO₂CH₃.

A fifth aspect of the invention relates to the preparation of a salt of vericiguat and trifluoroacetic acid.

A sixth aspect of the invention relates to the salt of vericiguat and trifluoroacetic acid, in particular the salt wherein vericiguat and trifluoroacetic acid are in a molar ratio of 1:1.

A seventh aspect of the invention relates to a process of preparing vericiguat from the trifluoroacetic acid salt of vericiguat.

### DESCRIPTION OF THE FIGURES

FIG. 1 shows the x-ray powder diffractogram (XRPD) obtained for the product 2-(5-fluoro-1-(2-fluorobenzyl)-1H-pyrazolo[3,4-b]pyridin-3-yl)-5-nitrosopyrimidin-4,6-diamine.

FIG. 2 shows the differential scanning calorimetry (DSC) graph obtained for the product 2-(5-fluoro-1-(2-fluorobenzyl)-1H-pyrazolo[3,4-b]pyridin-3-yl)-5-nitrosopyrimidin-4,6-diamine.

FIG. 3 shows the x-ray powder diffractogram (XRPD) obtained for the crystalline Form C of trifluoroacetic acid salt of vericiguat as obtained in example 6.

FIG. 4 shows the x-ray powder diffractogram (XRPD) obtained for the crystalline Form C of trifluoroacetic acid salt of vericiguat as obtained in example 6 after it has been maintained in a climatic chamber at 40° C. and 75% relative humidity.

### DETAILED DESCRIPTION OF THE INVENTION

In the context of the invention, the term “salt” should be understood to mean an ionic compound formed by a cation of the amino group of compounds of formula (I) or (II) or of vericiguat, and a counterion (an anion) such as, for example, the anion of an inorganic acid (such as, for example, hydrochloride, hydrobromide, hydroiodide, sulfate, nitrate, and phosphate, among others) or an organic acid (such as, for example, acetate, trifluoroacetate, maleate, fumarate, citrate, oxalate, succinate, tartrate, malate, mandelate, methanesulfonate, and p-toluenesulfonate, among others). Preferably, the salt of compound (II) is a hydrochloride and preferably the salt of vericiguat is a trifluoroacetate, more preferably the salt wherein vericiguat and trifluoroacetic acid are in a molar ratio of 1:1.

In the context of the present invention, the terms “approximate” and “about” in relation to a value refer to any value which is comprised in the interval defined by the value±5% of said value.

In the context of the present invention, the term “acid” refers to a substance capable of donating a proton (to a base). Said substance can be inorganic, such as in the case of hydrochloric, nitric, sulfuric, phosphoric, hydrobromic, and boric acids, or organic, such as in the case of formic, acetic, trifluoroacetic, propionic, oxalic, malic, maleic, fumaric, succinic, citric, tartaric, mandelic, methanesulfonic, p-toluenesulfonic, and benzoic acids.

In the context of the present invention, the term “base” refers to a substance capable of accepting a proton (from an acid). Said substance can be inorganic, such as in the case of alkali metal hydroxide salts and alkali and alkaline earth metal carbonate salts, or organic, such as in the case of pyridine, imidazole, and tertiary amines of formula NR₁R₂R₃, wherein each of R₁, R₂, or R₃ is a (C₁-C₆)alkyl group.

In the context of the present invention, the term “alkali metal” refers to a metal selected from the group consisting of lithium, sodium, potassium, rubidium, cesium, and francium. Preferably, the alkali metal is selected from the group consisting of lithium, sodium, and potassium; more preferably, it is sodium or potassium, and even more preferably, it is potassium.

### Compound of Formula (I) or a Salt Thereof

In a particular embodiment of the first aspect of the invention, the compound of formula (I) or the salt thereof is in solid form.

In a particular embodiment of the first aspect of the invention, the compound of formula (I) has an x-ray powder diffractogram measured with CuKα radiation comprising peaks at one or more of 6.7°, 16.2°, 21.0°, 21.4°, 26.3°, 28.5°, and 45.6° 2θ±0.2° 2θ.

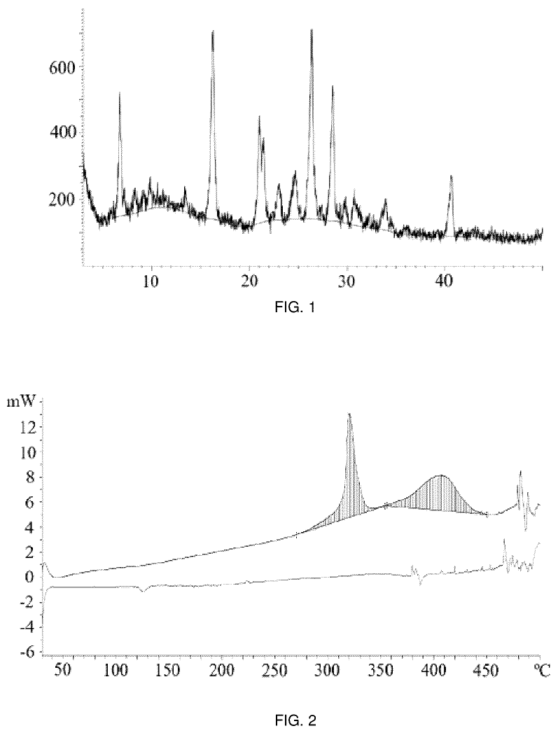

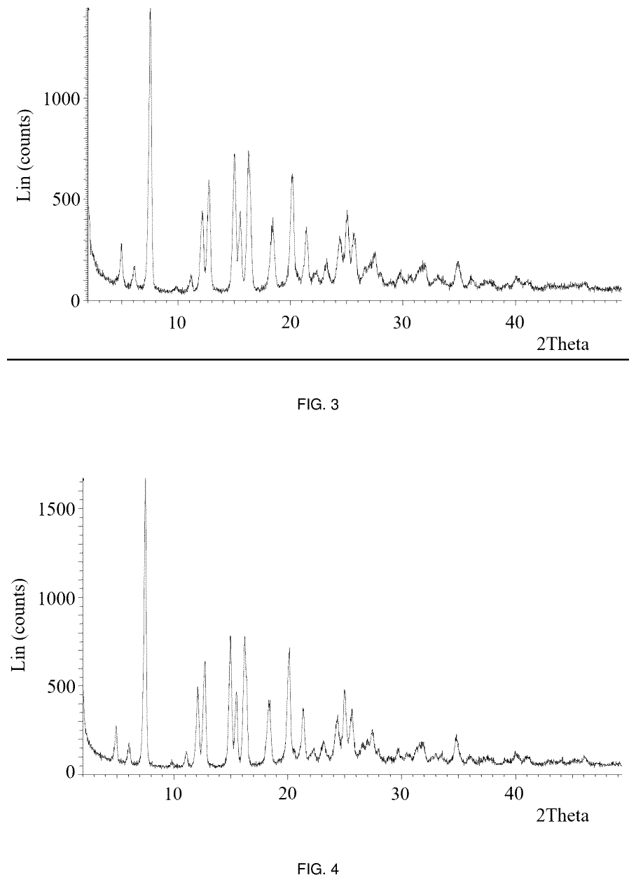

In a particular embodiment of the first aspect of the invention, the compound of formula (I) has an x-ray powder diffractogram measured with CuKα radiation essentially like the one shown in FIG. 1.

The X-ray diffractograms can be recorded using a powder diffraction system with a copper anode which emits CuKα radiation with a wavelength of 1.54 Å, in particular, following the method described in the examples.

In a particular embodiment of the first aspect of the invention, the compound of formula (I) presents a differential scanning calorimetry (DSC) diagram comprising an exothermic peak having a threshold temperature of about 315.9° C.±2° C. and another exothermic peak having a threshold temperature of about 368.6° C.±2° C.

In a particular embodiment of the first aspect of the invention, the compound of formula (I) presents a differential scanning calorimetry (DSC) diagram essentially like the one shown in FIG. 2.

The differential scanning calorimetry diagram can be obtained as described in the examples.

The threshold temperature or “T onset” refers to the temperature resulting from extrapolating the baseline before the start of transition and the baseline during energy absorption (tangent of the curve). It can be calculated as defined in the DIN ISO 11357-1:2016 (E) standard.

### Method for the Preparation of the Compound of Formula (I) or a Salt Thereof

As defined above, the second aspect of the invention relates to a method for the preparation of a compound of formula (I) or a salt thereof. Said method comprises reacting a compound of formula (II), which is 5-fluoro-1-(2-fluorobenzyl)-1H-pyrazolo[3,4-b]pyridin-3-carboximidamide, or a salt thereof, with a salt of formula (III)

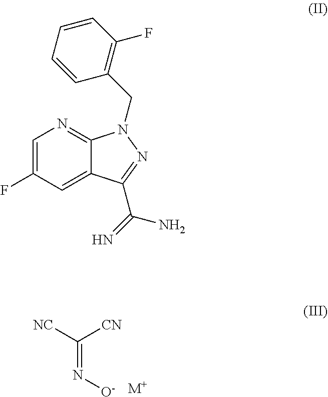

wherein M is an alkali metal.

In a preferred embodiment of the second aspect of the invention, the method for the preparation of the compound of formula (I) or a salt thereof comprises reacting a salt of the compound of formula (II) with a salt of formula (III). The compound of formula (II) comprises four nitrogen atoms which can be protonated to form salts. It is contemplated that between one and four nitrogen atoms are protonated in a salt of the compound of formula (II). The compound of formula (II) can form salts with several acids such as, for example, hydrochloric acid, acetic acid, hydrobromic acid, formic acid. As may be derived by one skilled in the art, a salt of the compound of formula (II) can be formed by reacting the compound of formula (II) with at least one equivalent of an acid as defined above, preferably between 1 and 4 equivalents. Preferably, said acid is hydrochloric acid.

In a preferred embodiment, the method for the preparation of the compound of formula (I) or a salt thereof comprises reacting a hydrochloride salt of the compound of formula (II) with a salt of formula (III). In said embodiment, the compound of formula (II) is a compound of formula (IIa)

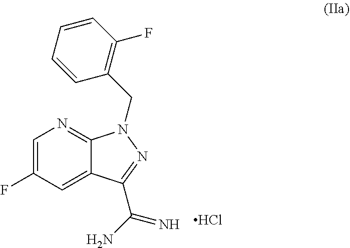 [SMILES: N=C(N)c1nn(Cc2ccccc2F)c2ncc(F)cc12 ; Cl]

In another preferred embodiment of the second aspect of the invention, the method for the preparation of the compound of formula (I) or a salt thereof comprises reacting a salt of the compound of formula (II) with a salt of formula (III), wherein M is sodium or potassium.

In another preferred embodiment of the second aspect of the invention, the method for the preparation of the compound of formula (I) or a salt thereof comprises reacting a salt of the compound of formula (II) with a salt of formula (III), wherein M is potassium.

In another preferred embodiment of the second aspect of the invention, the method for the preparation of the compound of formula (I) or a salt thereof comprises reacting a salt of the compound of formula (II) with a salt of formula (III) in the presence of a solvent. Any organic solvent suitable for carrying out the reaction can be used. Preferably, the solvent is an aprotic polar solvent.

In another preferred embodiment of the second aspect of the invention, the method for the preparation of the compound of formula (I) or a salt thereof comprises reacting a salt of the compound of formula (II) with a salt of formula (III) in the presence of a solvent selected from the group consisting of dimethylsulfoxide, N-methylpyrrolidone, N,N-dimethylformamide, N, N-dimethylacetamide, and acetonitrile.

In another preferred embodiment of the second aspect of the invention, the method for the preparation of the compound of formula (I) or a salt thereof comprises reacting a salt of the compound of formula (II) with a salt of formula (III) in the presence of a solvent which is N,N-dimethylformamide.

In another preferred embodiment of the second aspect of the invention, the method for the preparation of the compound of formula (I) or a salt thereof comprises reacting a salt of the compound of formula (II) with a salt of formula (III) at a temperature of between 80° C. and 160° C.

In another preferred embodiment of the second aspect of the invention, the method for the preparation of the compound of formula (I) or a salt thereof comprises reacting a salt of the compound of formula (II) with a salt of formula (III) at a temperature of between 120° C. and 140° C.

In another preferred embodiment of the second aspect of the invention, the method for the preparation of the compound of formula (I) or a salt thereof comprises reacting a salt of the compound of formula (II) with a salt of formula (III) at a temperature of about 130° C.

In another preferred embodiment of the second aspect of the invention, when the method for the preparation of the compound of formula (I) or a salt thereof comprises reacting a salt of the compound of formula (II) with a salt of formula (III) in the presence of a solvent as defined above, the concentration of the compound of formula (II) or the salt thereof in the solvent is comprised between 0.5 and 1 M.

In another preferred embodiment of the second aspect of the invention, when the method for the preparation of the compound of formula (I) or a salt thereof comprises reacting a salt of the compound of formula (II) with a salt of formula (III) in the presence of a solvent as defined above, the concentration of the compound of formula (II) or the salt thereof in the solvent is comprised between 0.7 M and 0.8 M. More preferably, the concentration of the compound of formula (II) or the salt thereof in the solvent is about 0.78 M.

In another preferred embodiment of the second aspect of the invention, the method for the preparation of the compound of formula (I) or a salt thereof comprises reacting a salt of the compound of formula (II) with a salt of formula (III), with the molar ratio of the compound of formula (II) or the salt thereof with respect to the salt of formula (III) being between 1:1 and 1:2, where values of 1:1 and 1:2 are included. Preferably, the salt of formula (III) is in excess with respect to the compound or the salt of formula (II).

In another preferred embodiment of the second aspect of the invention, the method for the preparation of the compound of formula (I) or a salt thereof comprises reacting a salt of the compound of formula (II) with a salt of formula (III), with the molar ratio of the compound of formula (II) or the salt thereof with respect to the salt of formula (III) being about 2:3.

In another preferred embodiment of the second aspect of the invention, the method for the preparation of the compound of formula (I) or a salt thereof which comprises reacting a salt of the compound of formula (II) with a salt of formula (III) is characterized by fulfilling two or more of the following conditions:
(i) the salt of the compound of formula (II) is a hydrochloride salt;
(ii) M is sodium or potassium in the salt of formula (III);
(iii) the reaction is carried out in the presence of an aprotic polar solvent, preferably in an amount sufficient for the concentration of the compound of formula (II) or the salt thereof in the solvent to be comprised between 0.5 and 1 M;
(iv) the molar ratio of the compound of formula (II) or the salt thereof with respect to the salt of formula (III) is between 1:1 and 1:2; and
(v) the method is carried out at a temperature of between 120° C. and 140° C.

In another preferred embodiment of the second aspect of the invention, the method for the preparation of the compound of formula (I) or a salt thereof which comprises reacting a salt of the compound of formula (II) with a salt of formula (III) is characterized by fulfilling two or more of the following conditions:
(i) the salt of the compound of formula (II) is a hydrochloride salt;
(ii) M is potassium in the salt of formula (III);
(iii) the reaction is carried out in the presence of a solvent which is N, N-dimethylformamide, preferably in an amount sufficient for the concentration of the compound of formula (II) or the salt thereof in the solvent to be comprised between 0.5 and 1 M;
(iv) the molar ratio of the compound of formula (II) or the salt thereof with respect to the salt of formula (III) is about 2:3; and
(v) the method is carried out at a temperature of between 120° C. and 140° C.

In another preferred embodiment of the second aspect of the invention, the method for the preparation of the compound of formula (I) or a salt thereof comprises isolating the compound of formula (I) or the salt thereof by means of precipitation; preferably from mixtures of water with N, N-dimethylformamide.

In another preferred embodiment of the second aspect of the invention, the compound of formula (II) is obtained through the steps of:
(a) preparing a suspension of 5-fluoro-1-(2-fluorobenzyl)-1H-pyrazolo[3,4-b]pyridine-3-carbonitrile in methanol at 0° C.±3° C.,
(b) adding sodium methoxide dropwise while keeping the temperature between 0 and 5° C.,
(c) allowing the resulting suspension to reach a temperature of 20° C.±5° C. and stirring the mixture during 12 to 24 hours,
(d) adding NH₄Cl and warming to a temperature of 45° C.±5° C. and stirred during 1 to 6 hours at said temperature,
(e) distilling off the solvent under vacuum at a temperature below 45° C.±5° C.,
(f) adding ethyl acetate and the mixture was maintained under stirring during 1 to 4 hours at a temperature of 20° C.±5° C.,
(g) distilling off the solvent under vacuum below 40° C.±5° C., and the reaction of compound of formula (II) or a salt thereof, with the salt of formula (III) is carried out without further isolation or purification of the product of step g).

### Use of the Compound of Formula (I) or a Salt Thereof in the Preparation of Vericiguat

As defined above, a third aspect of the invention relates to the use of the compound of formula (I) or a salt thereof in the preparation of vericiguat.

Method for the Preparation of Vericiquat from the Compound of Formula (I) or a Salt Thereof

A fourth aspect of the invention relates to a method for the preparation of vericiguat from the compound of formula (I) or a salt thereof. Said method comprises:
(a) reacting the compound of formula (I) or a salt thereof as defined in any of the particular and preferred embodiments of the first aspect of the invention in order to convert the nitrous group into an amino group; and
(b) reacting the compound resulting from step (a) in order to convert the amino group formed from the nitrous group of the compound of formula (I) or the salt thereof into a group of formula —NHCO₂CH₃.

In a preferred embodiment of the fourth aspect of the invention, reactions (a) and (b) are carried out in the same reaction medium.

In another preferred embodiment of the fourth aspect of the invention, reaction (b) is carried out without isolating the product of reaction (a). It has the advantage of the process being free of a purification step.

In another preferred embodiment of the fourth aspect, the invention relates to a method for the preparation of vericiguat from the compound of formula (I) or a salt thereof, wherein reactions (a) and (b) are carried out in the same reaction medium and reaction (b) is carried out without isolating the product of reaction (a). Said method advantageously allows vericiguat to be prepared in a single synthetic step from the compound of formula (I).

Any method suitable for converting the nitrous group of the compound of formula (I) or of the salt thereof into an amino group can be used for carrying out step (a). In a preferred embodiment of the fourth aspect of the invention, step (a) of the method for the preparation of vericiguat from the compound of formula (I) or from the salt thereof is carried out by means of catalytic hydrogenation. Catalytic hydrogenation methods are known in the art. Various catalysts such as, for example, rhodium, palladium, platinum, and nickel, can be used.

In a preferred embodiment of the fourth aspect of the invention, step (a) of the method for the preparation of vericiguat from the compound of formula (I) or from the salt thereof is carried out by means of catalytic hydrogenation using a palladium on carbon catalyst.

In another preferred embodiment of the fourth aspect of the invention, step (a) of the method for the preparation of vericiguat from the compound of formula (I) or from the salt thereof is carried out by means of catalytic hydrogenation using a hydrogen pressure of between 5 and 50 bars, preferably between 10 and 50 bars and/or at a temperature of between 30° C. and 60° C.

In another preferred embodiment of the fourth aspect of the invention, step (a) of the method for the preparation of vericiguat from the compound of formula (I) or from the salt thereof is carried out by means of catalytic hydrogenation using a hydrogen pressure of about 50 bars and/or at a temperature of about 60° C.

In another preferred embodiment of the fourth aspect of the invention, step (a) of the method for the preparation of vericiguat from the compound of formula (I) or from the salt thereof is carried out from the compound of formula (I).

In another preferred embodiment of the fourth aspect of the invention, step (a) of the method for the preparation of vericiguat from the compound of formula (I) or from the salt thereof is carried out by means of catalytic hydrogenation in the presence of an acid. It is particularly the case when step (a) of the method for the preparation of vericiguat uses the compound of formula (I) (and not a salt thereof) as a starting product. Said acid is preferably present in an amount of between 0.5 1.5 molar equivalents with respect to the compound of formula (I); more preferably between 0.5 and 1.1 molar equivalents, most preferably between 1.0 and 1.1 molar equivalents, and more preferably one molar equivalent.

In another preferred embodiment of the fourth aspect of the invention, step (a) of the method for the preparation of vericiguat from the compound of formula (I) or from the salt thereof is carried out by means of catalytic hydrogenation in the presence of trifluoroacetic acid. Said trifluoroacetic acid is preferably present in a sufficient amount of between 0.5 and 1.1 molar equivalents with respect to the compound of formula (I); preferably between 1 and 1.1 molar equivalents, and more preferably one molar equivalent (i.e., the molar ratio of the compound of formula (I) or the salt thereof with respect to the acid is about 1:1).

Step (a) of the fourth aspect of the invention can be carried out in the presence of a solvent suitable for this reaction. Examples of suitable organic solvents are C₁-C₄ alkanols such, as methanol, ethanol, n-propanol, isopropanol, n-butanol, sec-butanol, iso-butanol, tert-butanol, and mixture thereof; preferably methanol. It is contemplated that the concentration of the compound of formula (I) or the salt thereof in said solvent is between 0.1 and 1 M; preferably between 0.2 and 0.6 M.

In another particular embodiment of the fourth aspect of the invention, step (a) of the method for the preparation of vericiguat is carried out by means of catalytic hydrogenation and fulfills at least two, preferably all, of the following conditions:
(i) the catalyst is palladium on carbon;
(ii) the hydrogen pressure is about 50 bars and/or the temperature is about 60° C.;
(iii) step (a) is carried out based on the compound of formula (I) and in the presence of an acid;
(iv) the solvent is methanol;
(v) when step (a) is carried out based on the compound of formula (I) in the presence of an acid, said acid is trifluoroacetic acid; and
(vi) when step (a) is carried out in the presence of an acid, the molar ratio of the compound of formula (I) with respect to the acid is about 1:1.

In another particular embodiment of the fourth aspect of the invention, step (b) of the method for the preparation of vericiguat is carried out by reacting the compound resulting from step (a) with methyl chloroformate or dimethyl dicarbonate. Preferably, step (b) of the method for the preparation of vericiguat is carried out by reacting the compound resulting from step (a) with dimethyl dicarbonate.

Step (b) of the fourth aspect of the invention can be carried out in the presence of a solvent suitable for this reaction. Examples of suitable organic solvents are C₁-C₄ alkanols such as methanol, ethanol, n-propanol, isopropanol, n-butanol, sec-butanol, iso-butanol, tert-butanol, and mixture thereof; preferably methanol. It is contemplated that the concentration of the product of step (a) in said solvent is between 0.1 and 1 M; preferably between 0.2 and 0.6 M.

In another particular embodiment of the fourth aspect of the invention, step (b) of the method for the preparation of vericiguat is carried out at a temperature of about 60° C.

In another particular embodiment of the fourth aspect of the invention, step (b) of the method for the preparation of vericiguat is characterized in that the molar ratio of the compound resulting from step (a) with respect to dimethyl dicarbonate or methyl chloroformate is about 1:4

In another preferred embodiment of the fourth aspect of the invention, step (b) of the method for the preparation of vericiguat is carried out in the presence of an acid. Said acid is preferably present in an amount of between 0.5 and 1.5 molar equivalents with respect to the compound of formula (I); more preferably between 0.5 and 1.1 molar equivalent, most preferably between 1.0 and 1.1 molar equivalents, and more preferably one molar equivalent.

In another preferred embodiment of the fourth aspect of the invention, step (b) of the method for the preparation of vericiguat is carried out in the presence of trifluoroacetic acid. Said trifluoroacetic acid is preferably present in a sufficient amount of between 0.5 and 1.1 molar equivalents with respect to the compound of formula (I); preferably between 1 and 1.1 molar equivalents, and more preferably one molar equivalent (i.e., the molar ratio of the compound of formula (I) or the salt thereof with respect to the acid is about 1:1).

In another particular embodiment of the fourth aspect of the invention, step (b) of the method for the preparation of vericiguat is characterized in that it fulfills at least two of the following conditions:
(i) it is carried out by reacting the compound resulting from step (a) with dimethyl dicarbonate;
(ii) it is carried out at a temperature of about 60° C.;
(iii) it is carried out in the presence of a solvent which is methanol, and at a concentration of the product of step (a) of between 0.2 M and 0.6 M; and
(iv) the molar ratio of the product of step (a) with respect to dimethyl dicarbonate or methyl chloroformate is about 1:4.

In another particular embodiment of the fourth aspect of the invention, the method for the preparation of vericiguat from the compound of formula (I) or from the salt thereof is characterized in that reactions (a) and (b) are carried out in the same reaction medium and reaction (b) is carried out without isolating the product of reaction (a); and in that it fulfills at least one, preferably all, of the following conditions:
(i) step (a) is carried out by means of catalytic hydrogenation;
(ii) step (a) is carried out in the presence of a catalyst which is palladium on carbon;
(iii) step (b) is carried out based on the compound of formula (I) and in the presence of an acid;
(iv) when step (b) is carried out based on the compound of formula (I) in the presence of an acid, said acid is trifluoroacetic acid;
(v) when step (b) is carried out in the presence of an acid, the molar ratio of the compound of formula (I) with respect to the acid is about 1:1;
(vi) the hydrogen pressure is about 50 bars and/or the temperature is about 60° C.;
(vii) the solvent is methanol and the concentration of the compound of formula (I) or the salt thereof is between 0.2 M and 0.6 M;
(viii) the molar ratio of the compound of formula (I) or the salt thereof with respect to dimethyl dicarbonate or methyl chloroformate is about 1:4; and
(ix) step (b) is carried out by reacting the compound resulting from step (a) with dimethyl dicarbonate.

In another particular embodiment of the fourth aspect of the invention, the method for the preparation of vericiguat from the compound of formula (I) or from the salt thereof is characterized in that it further comprises step (c) of isolating vericiguat or the salt thereof formed in step (b). Any method can be used to isolate vericiguat or the salt thereof from the reaction medium of step (b).

In a particular embodiment, said step (c) is a step of precipitation in a solvent. Any solvent suitable for precipitating vericiguat can be used in said step. In a particular embodiment, said solvent is acetone.

Likewise, a preferred embodiment of the fourth aspect of the invention contemplates the following method:

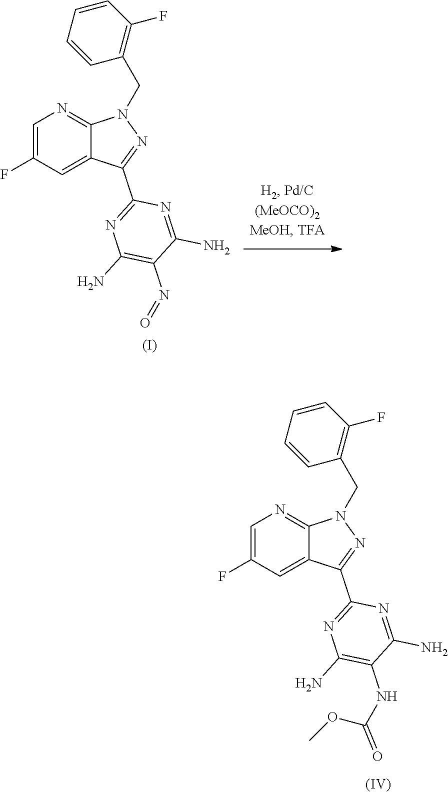

Said method advantageously allows vericiguat to be prepared in only two reaction steps from the compound of formula (II) or a salt thereof, which allows reducing the number of steps required for producing vericiguat with a high yield (95% for the last step, 79% from salt (11a)) and in a shorter period of time.

In a particular embodiment of the fourth aspect the starting compound of formula (IIa) may be obtained from compound of formula (V), i.e. 5-fluoro-1-(2-fluorobenzyl)-1H-pyrazolo[3,4-b]pyridine-3-carbonitrile:

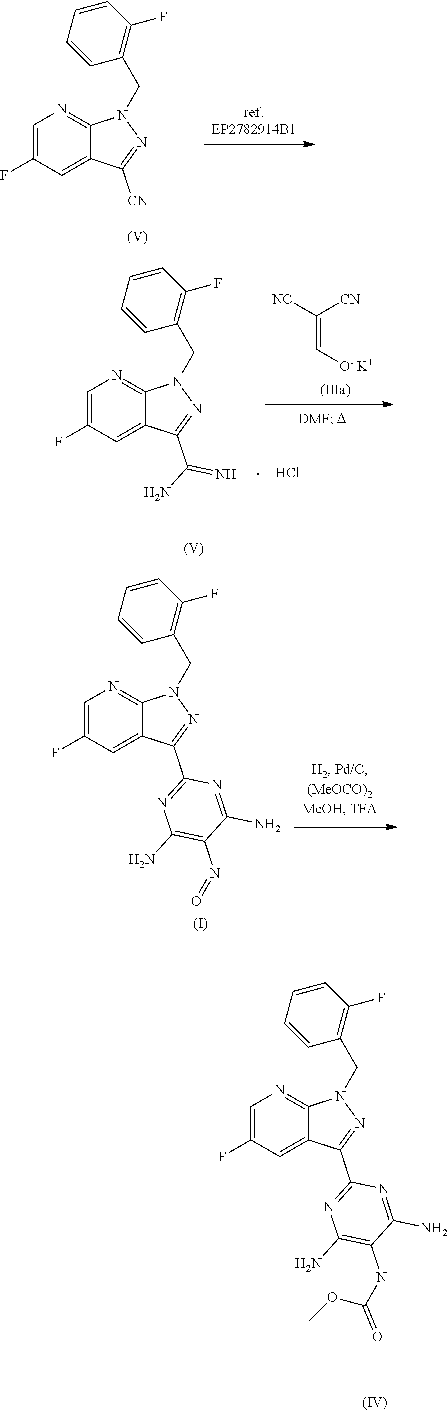

In a fifth aspect the invention relates to the preparation of acid addition salt of vericiguat and trifluoroacetic acid, more preferably the salt wherein vericiguat and trifluoroacetic acid are in a molar ratio of 1:1. The salt may be prepared by reacting compound of formula (I) as described in the fourth aspect of the invention (see paragraph to [081]) but using a sufficient amount of trifluoroacetic acid in the last reaction step which is between 1.5 and 2.5 molar equivalents with respect to the compound of formula (I); preferably between 2.0 and 2.1 equivalents, more preferably 2.0 equivalents (i.e. a molar ratio of compound (I) to trifluoroacetic acid is 1:2).

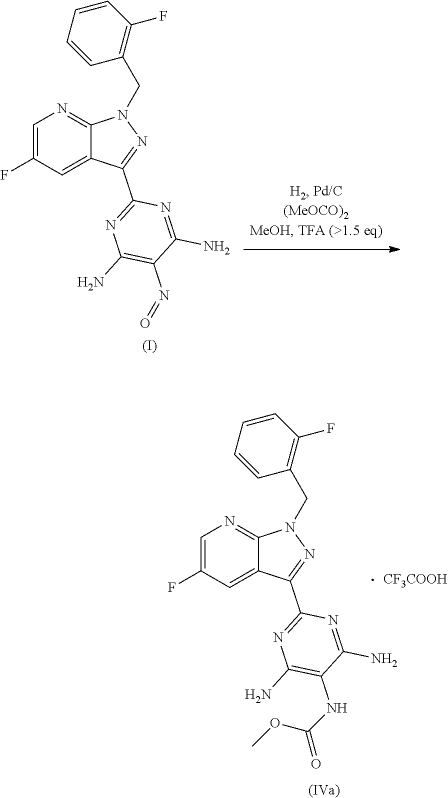

A sixth aspect of the invention relates to the salt of vericiguat and trifluoroacetic acid, in particular to the salt wherein vericiguat and trifluoroacetic acid are in a molar ratio of 1:1 in crystalline form C characterized by powder X-ray diffraction comprising the following °2θ (±0.2 °2θ) peaks: 7.5, 12.7, 15.0, 16.3 and 20.1 wherein the X-ray diffraction is measured at 25° C. using a CuKα radiation having a wavelength of 1.541838 Å, preferably having also the following 920 (±0.2 °2θ) peaks 4.9, 7.5, 12.1, 12.7, 15.0, 15.5, 16.3, 18.4, 20.1, 21.4, 24.4, 25.0 and 25.6 and having a water content between 1.5 and 3.5 wt %.

A seventh aspect of the invention relates to a process of preparing vericiguat from the trifluoroacetic acid salt of vericiguat.

In an embodiment of the seventh aspect the trifluoroacetic acid salt of 2-(5-fluoro-1-(2-fluorobenzyl)-1H-pyrazolo[3,4-b]pyridin-3-yl)-5-nitrosopyrimidin-4,6-diamine is solved or suspended in a solvent, preferably acetonitrile or a C₁₋₄ alkanol at a temperature ranging from 0 to 50° C., preferably about 20° C. To the suspension or solution a basic compound, selected from the group consisting of inorganic bases such as NaHCO₃, Na₂CO₃, KHCO₃, K₂CO₃, organic bases such as alkylamines, is added in a quantity ranging from 1 to 3 molar equivalents in relation to the trifluoroacetic acid salt. In a preferred embodiment NaHCO₃ is added in a quantity ranging from 1 to 2 molar equivalents in relation to the trifluoroacetic acid salt. After adding the base the mixture is brought to a temperature comprised between 20° about 40° C. and kept under stirring for a period comprised between 0.5 to 4 hours, preferably about 2 hours. The resulting mixture is slowly cooled to a temperature comprised between 20° C. and 50° C., preferably between 30° C. and about 40° C. and kept under stirring for a period of 0.5 to 6 hours, preferably about 2 hours at said temperature. The resulting mixture is slowly cooled to a temperature of between 0° C. to about 20° C., preferably about 20° C., and kept under stirring for a period of 0.5 to 2 hours, preferably 1 hour at said temperature. The resulting solid is filtered and washed with water. Then it is dried, for example in an air oven under vacuum at a temperature of about 50° C., to obtain a crystalline white solid corresponding to Form I of vericiguat having a powder X-ray diffraction as disclosed in the European patent EP 2 782 914 B1.

In another particular embodiment of the seventh aspect of the invention, the method for the preparation of vericiguat from the trifluoroacetic salt of vericiguat is characterized in that the reactions to obtain the trifluoroacetic acid salt of vericiguat and to obtain vericiguat from the trifluoroacetic acid salt thereof are carried out in the same reaction medium and the second reaction is carried out without isolating the trifluoroacetic salt of vericiguat obtained in the first reaction.

To facilitate the understanding of the preceding ideas, some examples of the experimental methods and embodiments of the present invention are described below. Said examples are non-limiting and merely illustrative.

### EXAMPLES

### Ultra-High Resolution Liquid Chromatography

The purity of the products obtained has been analyzed by means of the ultra-high resolution liquid chromatography technique in an Acquity model, Waters brand apparatus, provided with a photodiode detector, a mass detector, and a thermostatic oven for the column. A BEH C18 column (100×2.1 mm; 1.8 μm) and mobile phases A (25 mM of ammonium acetate, pH 5), B (acetonitrile), and C (water) were used with the following conditions of analysis:
Flow rate: 0.3 mL/min
Column temperature: 40° C.
Wavelength: 210 nm
Injection volume: 1 μL
Diluent: Acetonitrile/Water (1:1)
Gradient:

| t (min) | % A | % B | % C |
|---|---|---|---|
| 0 | 5 | 5 | 90 |
| 0.2 | 5 | 5 | 90 |
| 5 | 5 | 95 | 0 |
| 7.5 | 30 | 95 | 0 |
| 8 | 5 | 5 | 90 |
| 10 | 95 | 5 | 90 |

### Differential Scanning Calorimetry (DSC)

DSC analysis was performed in a Mettler Toledo 822e apparatus with STARe SW15 software using the following parameters: heating interval of 30 to 300° C. with a ramp of 10° C./min and an N₂ flow of 50 ml/min. The measurement is performed with a closed perforated capsule.

### Nuclear Magnetic Resonance

Proton nuclear magnetic resonance (¹H-NMR) and ¹³C-NMR analysis was performed in a 400 MHz Brucker Avance Ill spectrometer. The chemical shifts have been referenced to the DMSO-d6 signal (2.49 ppm for proton and 39.5 ppm for carbon).

### X-Ray Crystallography (XRPD)

XRPD analysis was performed using a BRUKER D2 PHASER model x-ray powder diffractometer equipped with a copper anode. The radiation used is CuKα with a wavelength of 1.54060 Å. The following scan parameters were used: 3-50° 2θ, continuous scan, ratio: 5.6°/minute.

The XRPD analysis of Form C of the salt of vericiguat with trifluoroacetic acid obtained in example 6 was performed using a Siemens model D-5000 X-ray poder diffractometer equipped with a copper anode. The radiation used is CuKα with a wavelength of 1.541838 Å. Scanning parameters: 4-50 degrees 2θ, continuous scan, ratio: 1.2 degrees/minute.

In one embodiment, the present invention contemplates that the compound of the invention is prepared and used in the preparation of vericiguat following the synthetic diagram below:

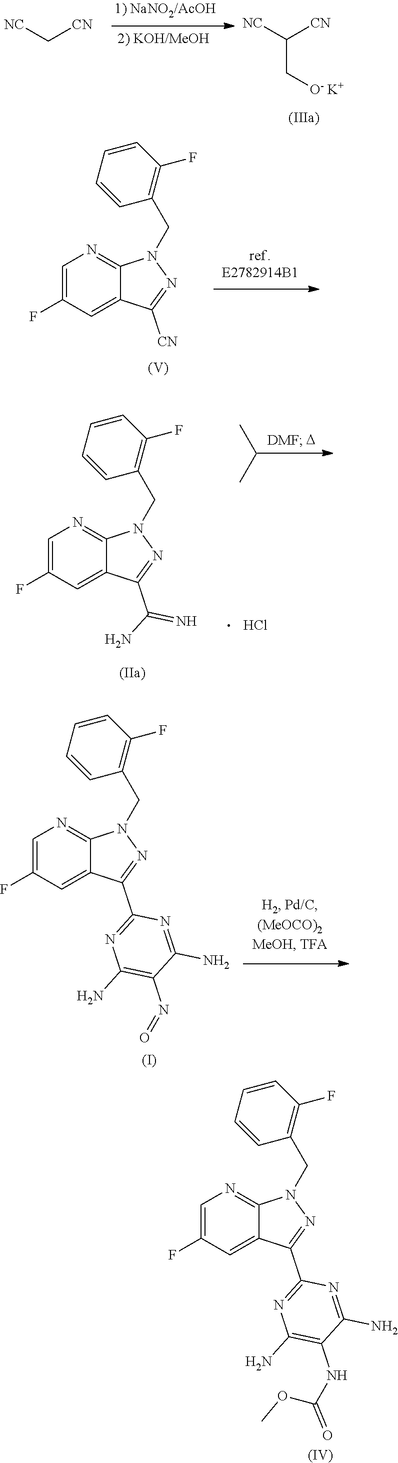

Compounds (V) and (11a) can be obtained by means of the method disclosed in the patent family corresponding to EP2782914B1 (ES2694158T3, Examples 8 and 9), incorporated by reference.

### Example 1. Obtaining the Hydroxyiminomalononitrile Potassium Salt (IIIa)

25 g (0.378 mol) of malonitrile were mixed with 55 mL of glacial acid acetic and 100 mL of water at a temperature of about 20° C. to obtain a solution which was cooled to a temperature of between −10 and −5° C. A previously prepared solution of 39.0 g (0.565 mol) of sodium nitrite in 60 mL of water was slowly added thereto, keeping the temperature between −10 and −5° C. The resulting mixture was kept under stirring at a temperature of between −10 and −5° C. for 45 minutes. The resulting mixture was then slowly added to 276 mL of an aqueous 2N hydrochloric acid solution and the resulting solution was kept under stirring for 15 minutes at a temperature of about 20° C. The reaction mixture was then extracted successively with 3 fractions of 200 mL of methyl-tert-butyl ether each. The resulting organic phases were all dried with anhydrous sodium sulfate and filtered through a filter with diatomaceous earth, then washed with an additional 200 ml of methyl-tert-butyl ether. The solvent of the resulting organic phase was removed by means of vacuum distillation to obtain a yellowish oil.

The oil thus obtained was slowly added at a temperature of between 0 and 5° C. to a solution previously prepared with 22.2 g (0.396 mol) of potassium hydroxide in 200 mL of methanol and cooled to a temperature of between 0 and 2° C. The mixture obtained was kept under stirring at a temperature of between 0 and 5° C. for 15 minutes, with the appearance of a precipitated solid being observed. Then, 500 mL of methyl-tert-butyl ether were added at a temperature of between 0 and 5° C. and the mixture thus obtained was kept under stirring for 2 hours at a temperature of between 0 and 2° C. The resulting solid was filtered and washed successively with two fractions of 25 mL of methyl-tert-butyl ether each. Lastly, it was dried in a vacuum oven at a temperature of 40° C. to obtain 38.83 g of a yellowish solid corresponding to hydroxyiminomalononitrile potassium salt (yield 77.1%).

### Example 2. Synthesis of 5-fluoro-1-(2-fluorobenzyl)-1H-pyrazolo[3,4-b]pyridine-3-carboximidamide hydrochloride (IIa)

100 g (359.5 mmol) of 5-fluoro-1-(2-fluorobenzyl)-1H-pyrazolo[3,4-b]pyridine-3-carbonitrile were suspended in 500 mL of methanol and cooled to about 0° C. 16.5 mL of sodium methoxide 25% in methanol (72.1 mmol) were added dropwise keeping the temperature between 0 and 5° C. The obtained yellow suspension was allowed to reach a temperature of about 20° C. and the mixture was stirred overnight.

21.2 g (393.3 mmol) of NH₄Cl were added and the mixture was warmed to a temperature of about 65° C. and stirred during 6 hours at said temperature. The solvent was distilled off under pressure, 200 mL of ethyl acetate were added and the mixture was maintained under stirring during two hours at a temperature of about 20° C. The solvent was distilled off under pressure, 400 mL of ethyl acetate were added and the mixture was maintained under stirring overnight at a temperature of about 20° C.

The resulting suspension was filtered to afford a solid that was washed four times with 50 mL each of ethyl acetate and dried with vacuum at about 50° C. to obtain 111.1 g (92.7% yield) of a white solid corresponding to 5-fluoro-1-(2-fluorobenzyl)-1H-pyrazolo[3,4-b]pyridine-3-carboximidamide hydrochloride (11a).

### Example 3. Obtaining 2-(5-fluoro-1-(2-fluorobenzyl)-1H-pyrazolo[3,4-b]pyridin-3-yl)-5-nitrosopyrimidin-4,6-diamine of formula (I)

18.0 g (0.056 mol) of 5-fluoro-1-(2-fluorobenzyl)-1H-pyrazolo[3,4b]pyridin-3-carboximidamide hydrochloride (11a) and 11.1 (0.083 mol) g of hydroxyiminomalononitrile potassium salt (IIIa) were mixed with 72 mL of DMF under N₂ atmosphere to obtain a solution at a temperature of about 20° C. The resulting solution was heated to a temperature of about 130° C. and kept under stirring at said temperature for 8 hours, with the total conversion of compound (11a) being observed.

The resulting mixture was then cooled to a temperature of between 9° and 100° C. and 15 ml of water were slowly added. The resulting mass was cooled to a temperature of about 20° C. and kept under stirring at said temperature for 3 hours. The resulting solid was filtered and washed successively with two fractions of 15 mL each of a 1:1 DMF/H₂O mixture, two fractions of 20 mL each of a 1:1 methanol/H₂O mixture, and 2 fractions of 20 mL each of methanol. Lastly, it was dried in an air oven at a temperature of 60° C. to obtain 17.10 g of a light cream-colored solid corresponding to 2-[5-fluoro-1-(2-fluorobenzyl)-1H-pyrazolo[3,4b]pyridin-3-yl]-5-nitrosopyrimidin-pyrimidin-4,6-diamine (yield 80.4% and UHPLC purity 98.8%).

¹H-NMR (d6-DMSO, 400 MHZ) δ(ppm): 10.16 (1H, d), 9.13 (1H, s), 8.99 (1H, dd), 8.76 (1H, d), 8.70 (1H, dd), 8.34 (1H, s), 7.37-7.33 (1H, m), 7.24-7.19 (2H, m), 7.16-7.12 (1H, m), 5.83 (2H, s)

¹³C-NMR (d6-DMSO, 100 MHZ) δ(ppm): 166.3, 164.1, 161.2, 158.7, 157.5, 155.0, 147.9, 146.6, 140.2, 139.4, 139.1, 130.2, 124.7, 123.4, 118.2, 115.5, 44.8 XRPD: 6.7° 2θ, 16.2° 2θ, 21.0° 2θ, 21.4° 2θ, 26.3° 2θ, 28.5° 2θ, and 45.6° 2θ, all of them with a margin of error of +0.2° 20. The x-ray powder diffractogram of the compound of formula (I) is shown in FIG. 1.

The differential scanning calorimetry (DSC) spectrum of the compound of formula (I) is shown in FIG. 2. In particular, the compound of formula (I) exhibits a differential scanning calorimetry (DSC) diagram comprising an endothermic peak having a threshold temperature of about 315.9° C. and another endothermic peak having a threshold temperature of about 368.6° C.

### Example 4. 2-[5-fluoro-1-(2-fluorobenzyl)-1H-pyrazolo[3,4-b]pyridin-3-yl]-5-nitroso-pyrimidine-4,6-diamine (I)

20 g (73.9 mmol) of 5-fluoro-1-(2-fluorobenzyl)-1H-pyrazolo[3,4-b]pyridine-3-carbonitrile were suspended in 120 mL of methanol and cooled to about 0° C. 3.4 mL of sodium methoxide 25% in methanol (14.9 mmol) were added dropwise keeping the temperature between 0 and 5° C. The obtained yellow suspension was allowed to reach a temperature of about 20° C. and the mixture was stirred overnight.

5.93 g (110.9 mmol) of NH₄Cl were added and the mixture was warmed to a temperature of about 45° C. and stirred during 2 hours at said temperature. The solvent was distilled off under pressure, 80 mL of ethyl acetate were added and the mixture was maintained under stirring during two hours at a temperature of about 20° C. The solvent was distilled off under pressure, 86 mL of DMF were added under N₂ atmosphere at a temperature of about 20° C. and 14.27 g (107.1 mmol) of (hydroxyimino) malononitrile sodium salt (IIIa) were further added. The mixture of reaction was heated at a temperature of about 120° C. and maintained for 7 hours at said temperature.

Once the maintenance was finished, the mixture of reaction was cooled to a temperature of about 90° C. 24 mL of water were slowly added. The obtained mixture was cooled to a temperature of about 20° C. and maintained under stirring during 2 hours at said temperature. The resulting suspension was filtered to afford a solid that was washed three times with 40 mL each time of a mixture of DMF/water 1:1 and two times with 40 mL each time of water and dried with vacuum at a temperature of about 50° C. to obtain 21.46 g (75.8% yield) of a white solid corresponding to 2-[5-fluoro-1-(2-fluorobenzyl)-1H-pyrazolo[3,4-b]pyridin-3-yl]-5-nitrosopyrimidine-4,6-diamine (I) with a purity of 99.0% UHPLC.

### Example 5. Obtaining methyl (4,6-diamino-2-[5-fluoro-1-(2-fluorobenzyl)-1H-pyrazol[3,4b]pyridin-3-yl)carbamate (vericiguat)

6.0 g (0.016 mol) of 2-(5-fluoro-1-(2-fluorobenzyl)-1H-pyrazolo[3,4-b]pyridin-3-yl)-5-nitrosopyrimidin-4,6-diamine (I) and 0.31 g of Pd/C 5% were mixed with 36 mL of methanol in a hydrogenation flask. Then, 6.9 mL (0.065 mol) of dimethyl dicarbonate and 1.2 mL of trifluoroacetic acid (0.016 mol) were added. Two successive inertization sequences were performed with N₂ and vacuum and, lastly, the inner pressure of the flask was adjusted to about 48 bars with H₂ atmosphere. The resulting mixture was heated to a temperature of about 60° C. and kept at said temperature and about 50 bars of H₂ pressure under stirring for 20 hours.

The reaction mixture was then cooled to a temperature of about 20° C. and depressurized and inertized with N₂. 0.33 g of carbon SX-1 were added, the resulting mixture was heated to about 65° C. and kept at said temperature under stirring for 30 minutes. The resulting mixture was filtered through a filter with diatomaceous earth, then washed with 36 mL of additional methanol. The solvent was removed by means of vacuum distillation to obtain a residue to which 66 mL of acetone were added. The resulting mixture was heated to a temperature of about 56° C. and kept under stirring for 10 minutes. The resulting mixture was slowly cooled to a temperature of about 20° C. and kept under stirring for 2 hours at said temperature. The resulting solid was filtered and washed with 2 successive fractions of 10 mL of acetone each. Lastly, it was dried in an air oven at a temperature of 60° C. to obtain 6.6 g of a virtually white solid corresponding to methyl (4,6-diamino-2-[5-fluoro-1-(2-fluorobenzyl)-1H-pyrazol[3,4b]pyridin-3-yl)carbamate (IV) (yield 98.6% and UHPLC purity 99.2%).

### Example 6. Obtaining the trifluoroacetic acid salt of methyl (4,6-diamino-2-[5-fluoro-1-(2-fluorobenzyl)-1H-pyrazol[3,4b]pyridin-3-yl)carbamate (vericiguat)

12 g (0.031 mol) of 2-(5-fluoro-1-(2-fluorobenzyl)-1H-pyrazolo[3,4-b]pyridin-3-yl)-5-nitrosopyrimidin-4,6-diamine (I) and 0.6 g of Pt/C 1% were mixed with 72 mL of methanol in a hydrogenation flask. Then, 13.5 mL (0.126 mol) of dimethyl dicarbonate and 4.75 ml of trifluoroacetic acid (0.062 mol) were added. Two successive inertization sequences were performed with N₂ and vacuum and, lastly, the inner pressure of the flask was adjusted to about 5 bars with H₂ atmosphere. The resulting mixture was heated to a temperature of about 60° C. and kept at said temperature and between 5 and 10 bars of H₂ pressure under stirring for 20 hours.

The reaction mixture was then cooled to a temperature of about 20° C. and depressurized and inertized with N₂. 0.6 g of carbon SX-1 and 300 mL of methanol were added, the resulting mixture was heated to about 60° C. and kept at said temperature under stirring for 30 minutes. The resulting mixture was filtered through a filter with diatomaceous earth, then washed with 50 mL of additional methanol. The solvent was removed by means of vacuum distillation to obtain a residue to which 20 mL of acetone were added. The solvent was again removed by means of vacuum distillation to obtain a residue to which 24 mL of dimethylformamide were added. The resulting mixture was heated to a temperature of about 80° C. and kept under stirring for 10 minutes. The resulting mixture was cooled to 60° C. and 32 mL of acetone were slowly added until a crystalline solid is formed. 112 mL of acetone were slowly added at a temperature of about 60° C. and the mixture was slowly cooled to a temperature of about 20° C. and kept under stirring for 1 hour at said temperature. The resulting solid was filtered and washed with 3 successive fractions of 5 mL of acetone each. Lastly, it was dried in an air oven at a temperature of 50° C. to obtain 14.76 g of a crystalline white solid corresponding to trifluoroacetic acid salt of methyl (4,6-diamino-2-[5-fluoro-1-(2-fluorobenzyl)-1H-pyrazol[3,4b]pyridin-3-yl)carbamate (yield 87.0% and UHPLC purity 99.3%). 1H-NMR (DMSO-d6, 400 MHZ) δ(ppm): 8.80-8.84 (2H, m), 8.34 (1H, broad s), 7.57 (4H, broad s), 7.35-7.40 (1H, m), 7.19-7.30 (1H, m), 7.10-7.19 (2H, m), 5.90 (2H, s), 3.66 (3H, s).

The product is characterized as Form C of vericiguat having a powder X-ray diffraction comprising the following °20 (+0.2 °20) peaks: 4.9, 7.5, 12.1, 12.7, 15.0, 15.5, 16.3, 18.4, 20.1, 21.4, 24.4, 25.0 and 25.6. It has a water content of 2.8 wt %.

The product has been maintained in a climatic chamber at 40° C. and 75% relative humidity and it has been observed that after 30 days no change occurs in the XRPD of the product as shown in FIG. 4. Similarly the water content remains stable at 2.9 wt % after 30 days at the above mentioned conditions.

### Example 7. Obtaining methyl (4,6-diamino-2-[5-fluoro-1-(2-fluorobenzyl)-1H-pyrazol[3,4b]pyridin-3-yl)carbamate (vericiguat)

5 g (9.25 mmol mol) of trifluoroacetic acid salt of 2-(5-fluoro-1-(2-fluorobenzyl)-1H-pyrazolo[3,4-b]pyridin-3-yl)-5-nitrosopyrimidin-4,6-diamine were suspended in 120 mL of acetonitrile at about 20° C. 1.0 g (11.9 mmol) of NaHCO₃ were added and the mixture was heated to a temperature of about 40° C. and kept under stirring for 2 hours. The resulting mixture was slowly cooled to a temperature of about 20° C. and kept under stirring for 1 hour at said temperature. The resulting solid was filtered and washed with 3 successive fractions of 5 mL of water each. It was dried in an air oven under vacuum at a temperature of about 50° C. to obtain 3.51 g of a crystalline white solid corresponding to methyl (4,6-diamino-2-[5-fluoro-1-(2-fluorobenzyl)-1H-pyrazol[3,4b]pyridin-3-yl)carbamate (IV) (yield 89.1% and UHPLC purity 99.4%). The product is characterized as Form I of vericiguat having a powder X-ray diffraction as disclosed in the European patent EP 2 782 914 B1.

### Example 8. Obtaining methyl (4,6-diamino-2-[5-fluoro-1-(2-fluorobenzyl)-1H-pyrazol[3,4b]pyridin-3-yl)carbamate (vericiguat)

5 g (9.25 mol) of trifluoroacetic acid salt of 2-(5-fluoro-1-(2-fluorobenzyl)-1H-pyrazolo[3,4-b]pyridin-3-yl)-5-nitrosopyrimidin-4,6-diamine were suspended in 100 ml of ethanol at about 20° C. 1.4 ml of triethylamine were added and the mixture was heated to a temperature of about 40° C. and kept under stirring for 2 hours. The resulting mixture was slowly cooled to a temperature of about 20° C. and kept under stirring for 1 hour at said temperature. The resulting solid was filtered and washed with 3 successive fractions of 5 mL of ethanol each and 3 successive fractions of 5 ml of water each. It was dried in an air oven under vacuum at a temperature of about 50° C. to obtain 3.48 g of a crystalline white solid corresponding to methyl (4,6-diamino-2-[5-fluoro-1-(2-fluorobenzyl)-1H-pyrazol[3,4b]pyridin-3-yl)carbamate (IV) (yield 88.3% and UHPLC purity 99.1%). The product is characterized as Form I of vericiguat having a powder X-ray diffraction as disclosed in the European patent EP 2 782 914 B1.

### Example 9. Obtaining the hydroxyiminomalononitrile sodium salt (IIIb)

20 g (0.303 mol) of malonitrile were mixed with 44 mL of glacial acid acetic and 80 mL of water at a temperature of about 20° C. to obtain a solution which was cooled to a temperature of between-5 and 0° C. A previously prepared solution of 31.16 g (0.452 mol) of sodium nitrite in 47 mL of water was slowly added thereto, keeping the temperature between-5 and 0° C. The resulting mixture was kept under stirring at a temperature of about 0° C. for 1 hour.

The resulting mixture was then slowly added to 220 mL of an aqueous 2N hydrochloric acid solution and the resulting solution was kept under stirring for 15 minutes at a temperature of about 20° C. It was then extracted successively with 3 fractions of 250 mL each of methyl-tert-butyl ether. The solvent of the resulting organic phase was removed by means of vacuum distillation to obtain a yellowish oil which was dissolved in 100 ml of dichloromethane. The solvent of the resulting solution was removed by means of vacuum distillation to obtain a yellowish oil.

The oil thus obtained was dissolved in 150 mL of methanol and the solution obtained was cooled to a temperature of about 0° C. 57.06 g of a sodium methoxide solution in 30 wt % methanol was slowly added to said solution at a temperature of between 0 and 5° C. The mixture thus obtained was kept under stirring at a temperature of between 0 and 5° C. for 15 minutes, with the appearance of a precipitated solid being observed. Then, 400 mL of methyl-tert-butyl ether were added at a temperature of between 0 and 5° C. and the mixture thus obtained was kept under stirring for 4 hours at a temperature of between 0 and 5° C. The resulting solid was filtered and washed successively with four fractions of 25 mL each of methyl-tert-butyl ether. Lastly, it was dried in a vacuum oven at a temperature of 40° C. to obtain 22.6 g of a yellowish solid corresponding to the hydroxyiminomalononitrile sodium salt (IIIb) (yield 63.8%).

### Example 10. Obtaining 2-[5-fluoro-1-(2-fluorobenzyl)-1H-pyrazolo[3,4b]pyridin-3-yl]-5-nitro-phenyldiazenyl]-pyrimidin-4,6-diamine (I)

10.0 g (0.031 mol) of 5-fluoro-1-(2-fluorobenzyl)-1H-pyrazolo[3,4b]pyridin-3-carboximidamide hydrochloride (11a) and 5.45 (0.047 mol) g of hydroxyiminomalononitrile sodium salt (IIIb) were mixed with 40 mL of N,N-dimethylformamide under N₂ atmosphere to obtain a solution at a temperature of about 20° C. The resulting solution was heated to a temperature of about 130° C. and kept under stirring at said temperature for 9 hours, with the total conversion of compound (11a) being observed.

The resulting mixture was then cooled to a temperature of between 9° and 100° C. and 8 mL of water were slowly added. The resulting mass was cooled to a temperature of about 20° C. and kept under stirring at said temperature for 2 hours. The resulting solid was filtered and washed successively with two fractions of 10 mL each of a 1:1 (vol/vol) DMF/H₂O mixture, a fraction of 10 ml of water, two fractions of 15 mL each of a 1:1 (vol/vol) methanol/H₂O mixture, and 2 fractions of 15 mL each of methanol. Lastly, it was dried in an air oven at a temperature of 50° C. to obtain 8.30 g of a light cream-colored solid corresponding to 2-[5-fluoro-1-(2-fluorobenzyl)-1H-pyrazolo[3,4b]pyridin-3-yl]-5-nitro-phenyldiazenyl]-pyrimidin-4,6-diamine (yield 70.2% and UHPLC purity 97.8%).

The data from ¹H-NMR (de-DMSO, 400 MHZ), ¹³C-NMR (de-DMSO, 100 MHZ)· (ppm), XRPD, and DSC of the solid obtained coincides with that of the solid obtained following the methodology of Example 2.

8.0 g of the solid thus obtained corresponding to 2-[5-fluoro-1-(2-fluorobenzyl)-1H-pyrazolo[3,4b]pyridin-3-yl]-5-nitro-phenyldiazenyl]-pyrimidin-4,6-diamine were transformed into vericiguat (8.71 g) following the methodology of Example 3 (yield 97.6% and UHPLC purity 99.3%).

1. A compound of formula (I), which is 2-(5-fluoro-1-(2-fluorobenzyl)-1H-pyrazolo[3,4-b]pyridin-3-yl)-5-nitrosopyrimidin-4,6-diamine, or a salt thereof

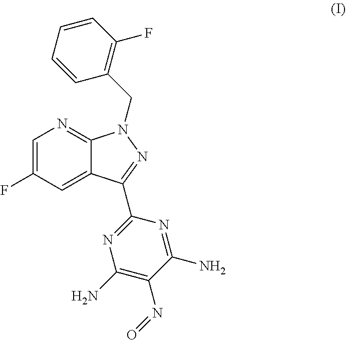 [SMILES: Nc1nc(-c2nn(Cc3ccccc3F)c3ncc(F)cc23)nc(N)c1N=O]

2. The compound according to claim 1, characterized in that:
a) it has an x-ray powder diffractogram measured with CuKα radiation comprising peaks at 6.7° 2θ, 16.2° 2θ, 21.0° 2θ, 21.4° 2θ, 26.3° 2θ, 28.5° 2θ, and 45.6° 2θ, all of them with a margin of error of ±0.2° 2θU;
b) it has an x-ray powder diffractogram measured with CuKα radiation essentially like the one shown in FIG. 1;
c) it has a differential scanning calorimetry (DSC) diagram comprising an exothermic peak having a threshold temperature of about 315.9° C.±2° C. and another exothermic peak having a threshold temperature of about 368.6±2° C.; and/or
d) it has a differential scanning calorimetry (DSC) diagram essentially like the one shown in FIG. 2.

3.-5. (canceled)

6. A method for the preparation of a compound of formula (I) or a salt thereof as defined in claim 1, characterized in that it comprises reacting a compound of formula (II), which is 5-fluoro-1-(2-fluorobenzyl)-1H-pyrazolo[3,4-b]pyridin-3-carboximidamide, or a salt thereof, with a salt of formula (III) 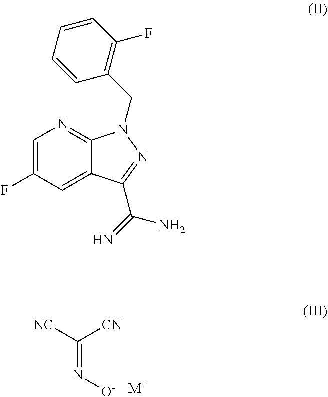
wherein M is an alkali metal.

7. (canceled)

8. The method according to claim 6, characterized in that it comprises reacting a hydrochloride salt of the compound of formula (II) with a salt of formula (III).

9. (canceled)

10. The method according to claim 6, characterized in that M is potassium in the salt of formula (III).

11. The method according to claim 6, characterized in that it is carried out in the presence of a solvent.

12.-17. (canceled)

18. The method according to claim 6, characterized in that it comprises reacting a hydrochloride salt of the compound of formula (II) with a salt of formula (III) wherein M is potassium, and in that it is carried out: (i) in the presence of a solvent which is N,N-dimethylformamide; (ii) at a temperature of between 120° C. and 140° C.; (iii) at a concentration of the compound of formula (II) or the salt thereof of between 0.5 and 1 M, and (iv) at a molar ratio of the hydrochloride of the compound of formula (II) with respect to the salt of formula (III) of about 2:3.

19. (canceled)

20. A method for the preparation of vericiguat, which is methyl (4,6-diamino-2-(5-fluoro-1-(2-fluorobenzyl)-1H-pyrazolo[3,4-b]pyridin-3-yl)pyrimidin-5-yl)carbamate, or a salt thereof, which comprises:
(a) reacting the compound of formula (I) or a salt thereof as defined in claim 1 in order to convert the nitrous group into an amino group; and
(b) reacting the compound resulting from step (a) in order to convert the amino group formed from the nitrous group of the compound of formula (I) or the salt thereof into a group of formula —NHCO₂CH₃.

21. The method according to claim 20, characterized in that reactions (a) and (b) are carried out in the same reaction medium.

22. The method according to claim 20, characterized in that reaction (b) is carried out without isolating the product of reaction (a).

23.-25. (canceled)

26. The method according to claim 20, characterized in that step (b) is carried out in the presence of an acid.

27. (canceled)

28. The method according to claim 26, characterized in that the molar ratio of the compound of formula (I) or the salt thereof with respect to the acid is about 1:1.

29. The method according to claim 20, characterized in that step (b) is carried out by reacting the compound resulting from step (a) with dimethyl dicarbonate.

30.-31. (canceled)

32. The method according to claim 20, characterized in that it is carried out in the presence of a solvent.

33.-34. (canceled)

35. The method according to claim 20, comprising step (c) of isolating vericiguat or the salt thereof formed in step (b).

36.-42. (canceled)

43. A method for the preparation of the trifluoroacetic acid salt of vericiguat, which comprises:
(a) reacting the compound of formula (I) or a salt thereof as defined in claim 1 in order to convert the nitrous group into an amino group; and
(b) reacting the compound resulting from step (a) in order to convert the amino group formed from the nitrous group of the compound of formula (I) or the salt thereof into a group of formula —NHCO₂CH₃, wherein step (b) is carried out in the presence of trifluoroacetic acid and wherein the molar ratio of the compound of formula (I) or the salt thereof with respect to trifluoroacetic acid is between 1.5 and 2.5 molar equivalents with respect to the compound of formula (I).

44. The method according to claim 43, characterized in that reactions (a) and (b) are carried out in the same reaction medium.

45.-48. (canceled)

49. The method according to claim 43, characterized in that the molar ratio of the compound of formula (I) or the salt thereof with respect to trifluoroacetic acid is about 2:0.

50. The method according to claim 43, characterized in that step (b) is carried out by reacting the compound resulting from step (a) with dimethyl dicarbonate.

51.-52. (canceled)

53. The method according to claim 43, characterized in that it is carried out in the presence of a solvent.

54.-55. (canceled)

56. The method according to claim 43, comprising step (c) of isolating the trifluoroacetic acid salt vericiguat or the salt thereof formed in step (b).

57.-58. (canceled)

59. The method according to claim 43, characterized in that step (a) is carried out in the presence of an acid.

60.-61. (canceled)

62. The method according to claim 43 comprising step c) of treating the trifluoroacetic acid salt of vericiguat with a base to obtain vericiguat.
